(19) United States
(12) Patent Application Publication
(10) Pub. No.: US 2015/0329274 A1
(43) Pub. Date: Nov. 19, 2015

(54) GERM SHIELD KIT

(71) Applicant: Jadex Ventures, LLC, Nashville, TN (US)

(72) Inventors: Jessica Wilbanks Bennett, Loganville, GA (US); Valerie J. Coles, Snellville, GA (US)

(21) Appl. No.: 14/712,408

(22) Filed: May 14, 2015

Related U.S. Application Data
(60) Provisional application No. 61/993,040, filed on May 14, 2014.

(51) Int. Cl. B65D 85/00; B64D 11/06

(52) U.S. Cl. CPC B65D 85/00; B64D 11/06

(57) ABSTRACT

Embodiments of a germ shield kit are described. In one embodiment, the germ shield kit includes a headrest cover, a tray cover, and at least one disinfectant wipe. In other embodiments, the kit may include additional barrier or comfort articles such as slippers, socks, eye covers, ear buds, or other items. By way of use of the germ shield kit, the general public may be able to reduce the likelihood of being exposed to such germs or bacteria while traveling, for example.

### CROSS-REFERENCE TO RELATED APPLICATIONS

This application claims the benefit of U.S. Provisional Patent Application No. 61/993,040, entitled “GERM SHIELD KIT,” and filed May 14, 2014, which is hereby incorporated herein by reference in its entirety.

### BACKGROUND

Today, individuals are traveling more frequently for business, visiting family, vacation, and other reasons. To some extent, the increased national and international travel of individuals has resulted in the wider spread of germs, bacteria, and other microorganisms which spread disease or sickness. In the same context, while individuals are traveling, they may be more prone to sickness due to exhaustion, dehydration, poor diet, or other physical and/or environmental factors.

### BRIEF DESCRIPTION OF THE DRAWINGS

For a more complete understanding of the embodiments described herein and the advantages thereof, reference is now made to the following description, in conjunction with the accompanying figures briefly described as follows:

FIG. 1A illustrates example packaging for a germ shield kit according to various embodiments described herein.

FIG. 1B illustrates other example packaging for the germ shield kit according to various embodiments described herein.

FIG. 2 illustrates an example of the content of the germ shield kit of FIG. 1.

FIG. 3 illustrates an example headrest cover of the germ shield kit of FIG. 1 according to an example embodiment.

FIG. 4 illustrates an example disinfectant wipe pouch and disinfectant wipe of the germ shield kit of FIG. 1 according to an example embodiment.

FIG. 5A illustrates a tray cover of the germ shield kit of FIG. 1 according to an example embodiment.

FIG. 5B illustrates a tray cover of the germ shield kit of FIG. 1 according to another example embodiment.

The drawings illustrate only example embodiments and are therefore not to be considered limiting of the scope described herein, as other equally effective embodiments are within the scope of this disclosure. The elements and features shown in the drawings are not necessarily drawn to scale, but emphasis is instead placed upon clearly illustrating the principles of the embodiments. Additionally, certain dimensions may be exaggerated to help visually convey certain principles. In the drawings, similar reference numerals between figures designate like or corresponding, but not necessarily the same, elements.

### DETAILED DESCRIPTION

It has been documented by governmental authorities, health advocacy groups, and the media that airline seats (i.e., seats on an airplane) and the area surrounding airline seats are more likely to harbor germs, bacteria, or other microorganisms that spread or cause disease or sickness. In this context, according to aspects of the embodiments described herein, using a combination of sanitary wipes and barriers, the general public may be able to reduce the likelihood of being exposed to such germs or bacteria while traveling. In one embodiment, a germ shield kit is described. The germ shield kit includes a headrest cover, a tray cover, and at least one disinfectant wipe. In other embodiments, the kit may include additional barrier or comfort articles such as slippers, socks, eye covers, ear buds, or other items.

In the following paragraphs, embodiments of a germ shield kit are described in detail by way of example with reference to the attached drawings. The embodiments are not limited in application, however, to the examples set forth herein. Turning to the drawings below, a general description of exemplary embodiments of a germ shield kit and the respective articles of the germ shield kit are provided.

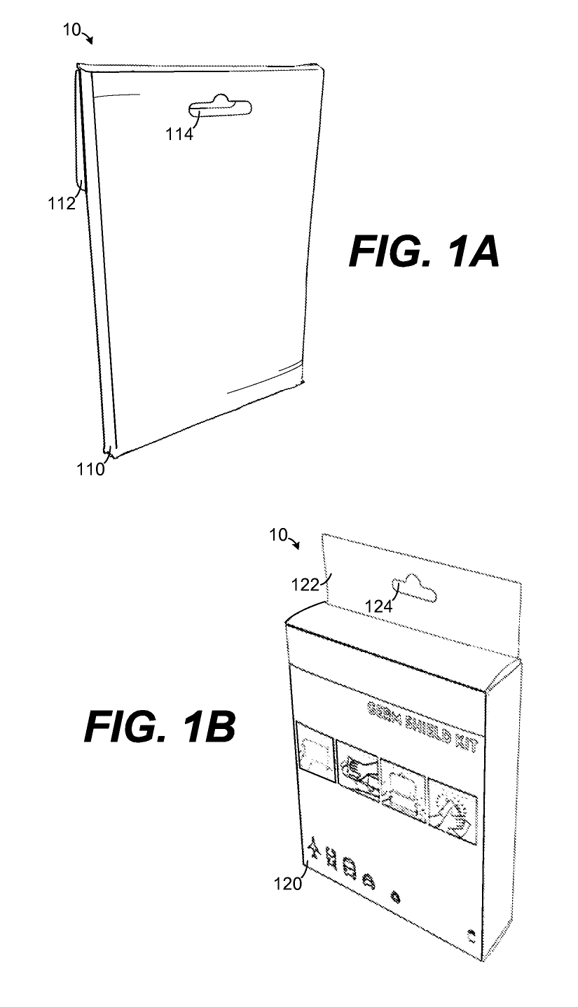

FIG. 1 illustrates example packaging 110 for a germ shield kit 10 according to various embodiments described herein. The packaging 110 for the germ shield kit 10 may be formed from any suitable type of material, such as plastic or paper, for example. The packaging 110 is generally provided to contain or enclose the articles of the germ shield kit 10 as they are described herein. In one embodiment, the packaging 110 includes a flap 112 which may be relied upon to open and close the packaging 110. As the packaging 110 is designed to be suitable for presentation in retail sales environments, the packaging 110 may include a packaging hole 114. The packaging hole 114 may be relied upon to hang the packaging 110 on a shelving system at, for example, a retail kiosk, store, or other outlet. It should be appreciated that the packaging 110 illustrated in FIG. 1 is provided by way of example only, as any suitable type of retail packaging may be relied upon among the embodiments described herein. In other words, the packaging 110 may be embodied as any suitable type of retail packaging that contains or encloses the articles of the germ shield kit 10 described herein.

As another example of packaging for the germ shield kit 10, FIG. 1B illustrates packaging 120. Similar to the packaging 110, the packaging 120 may be formed from any suitable type of material, such as plastic or paper, for example. The packaging 120 is also provided to contain or enclose the articles of the germ shield kit 10. As illustrated, the packaging 120 is embodied as a rectangular box having dimensions of about 4 inches in width, 4 inches in length, and 1 inch thick. The size of the packaging 120 may vary, however, based on the number of articles in the germ shield kit 10, for example. In one embodiment, the packaging 120 includes a tab 122 and a packaging hole 124. The packaging hole 124 may be relied upon to hang the packaging 120, for example, a retail kiosk, store, or other outlet.

As described in further detail below, the articles of the germ shield kit 10 may be primarily used as either or both a disinfectant for or as a shield against germs, bacteria, or other microorganisms that spread or cause disease or sickness (i.e., germ shield articles). While the germ shield kit 10 is described below for use in connection with airline transportation, the germ shield kit 10 may be used in other circumstances, such as in restaurants, movie theaters, rental cars, taxicabs, subways, or cruise ships, for example.

FIG. 2 illustrates an example of the contents of the germ shield kit 10 of FIG. 1. As illustrated, the germ shield kit 10 includes a first sealed package 200 of articles and a second sealed package 202 of articles. Here, it is noted that the packaging 110 may be sized to carry two sealed packages, where each sealed package includes articles for use by an individual on one flight. Thus, for a round trip, the packaging 110 may be sized to carry a sealed package for both outbound and inbound flights. In other embodiments, the germ shield kit 10 may include fewer or greater sealed packages of articles. The first sealed package 200 may be embodied as a bag 210. In one embodiment, within the bag 210, the germ shield kit 10 includes a headrest cover 220, a tray cover 230, and at least one disinfectant wipe pouch 240. In other embodiments, the bag 210 may include a plurality of headrest covers, tray covers, and disinfectant wipe pouches.

The bag 210 may be embodied as a sealed plastic bag. For example, the bag 210 may be sealed using heat, adhesives, or any other suitable means. Because the headrest cover 220, tray cover 230, and at least one disinfectant wipe pouch 240 may be manufactured and packaged (in the bag 210) in a clean or sterile environment, the contents of the bag 210 may be substantially free from germs, bacteria, or other microorganisms. In some embodiments, the bag 210 may be vacuum sealed to reduce the overall size of the bag 210, making it substantially free of air.

In one embodiment, the bag 210 may be expandable. In other words, once the bag 210 is opened, the bag 210 may itself expand beyond the size which was required to hold the headrest cover 220, the tray cover 230, and the at least one disinfectant wipe pouch 240. Thus, even after the headrest cover 220, the tray cover 230, and the at least one disinfectant wipe pouch 240 are opened and/or unfolded, each may be placed back into the bag 210 for disposal. In this sense, the bag 210 may be relied upon for the disposal of the items sealed within it, after use.

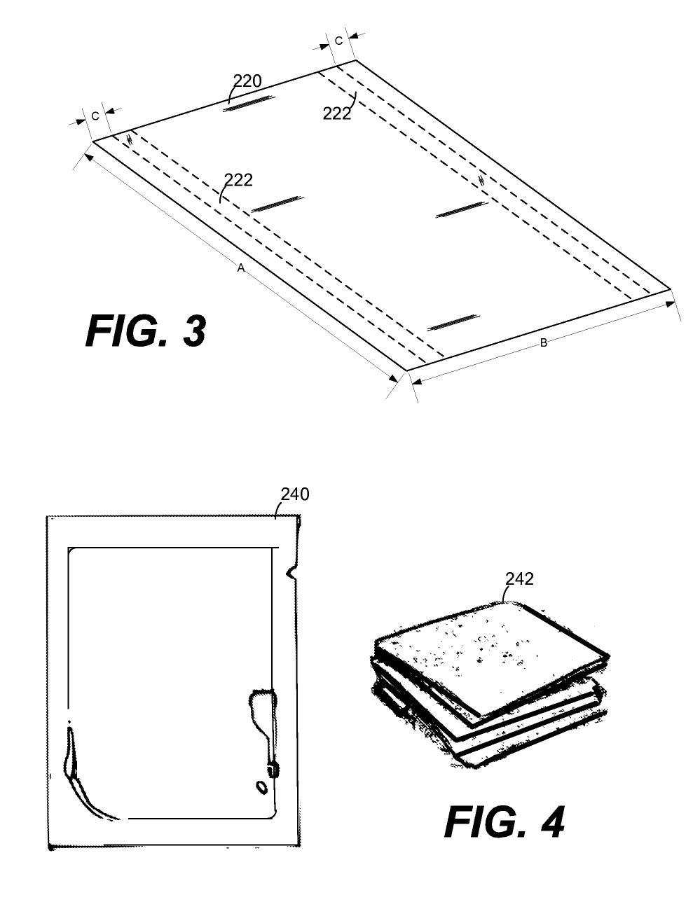

FIG. 3 illustrates an example headrest cover 220 of the germ shield kit 10 of FIG. 1 according to an example embodiment. As illustrated in FIG. 3, the headrest cover 220 may be embodied as a rectangle-shaped fabric material having length and width dimensions of A and B, respectively. In one exemplary embodiment, based on the size of an average headrest of a seat on an airplane, the length A of the headrest cover 220 may be selected at about 20 inches, and the width B of the headrest cover 220 may be selected at about 16 inches. It should be appreciated, however, that the length and width dimensions of the headrest cover 220 may vary among embodiments. In various embodiments, the headrest cover 220 may be shaped as a square, circle, ellipse, or other shapes. In certain embodiments, the headrest cover 220 may be cut to take the shape of an animal, character, vehicle, plant, building, or other shape, for example, especially if the germ shield kit 10 is marketed for children or parents.

The headrest cover 220 may include longitudinal adhesive strips 222, each positioned from the lengthwise edges of the headrest cover 220 by the distance C. The adhesive strips 222 may be about 1 inch wide, although other widths are within the scope of the embodiments described herein. In various embodiments, the distance C may range from ½ to 1 inch, for example. In FIG. 3, two adhesive strips 222 along the lengthwise edges of the headrest cover 220 are illustrated. In other embodiments, adhesive strips may be provided along only one lengthwise edge, along the widthwise (and not lengthwise) edges, along only one widthwise (and not lengthwise) edge, or along both the lengthwise and widthwise edges. The type of adhesive used for the adhesive strips 222 may be selected for suitable adherence to any of the materials commonly used to cover headrests of airplane seats. Thus, the adhesive used for the adhesive strips 222 may be selected for suitable adherence to fabric, plastic, leather, or vinyl, for example. In another aspect, the adhesive may be selected so as to be removable without leaving adhesive residue. The adhesive strips 222 may be relied upon to ensure a stable bond while also being easily removable, repositionable, and replaceable without leaving adhesive residue. In other embodiments, the headrest cover 220 may include one or more hook fastener (e.g., VELCRO®) strips or elastic bands or strips for securing the headrest cover 220 to an airplane headrest.

The headrest cover 220 may be formed from a fabric material such as a non-woven SMS fabric, for example, although the use of other materials is within the scope of the embodiments described herein. Among other factors, the fabric material of the headrest cover 220 may be generally selected based on one or more of the following example factors: resistance to tears and punctures, anti-static properties, barrier properties, softness, and soundlessness. In this context, the fabric material of the headrest cover 220 may be formed from any suitable base material, such as paper, paper pulp, polymers, plastics (e.g., polypropylene), or any combination thereof. In various embodiments, the headrest cover 220 may be formed using a bi- or tri-laminate (i.e., layer) construction to provide relatively high tensile strength, while maintaining a soft and drapable texture. Based on its composition, the headrest cover 220 may provide a fluid and particulate barrier against germs, bacteria, or other microorganisms.

In use, the headrest cover 220 may be draped over a headrest of an airplane seat to provide a barrier between the headrest area of the airplane seat and an individual's head, neck, back, hair, etc. Before being placed and secured to the headrest area, a paper backing (e.g., waxed or slicked paper backing) is removed from the adhesive strips 222. After the paper backing is removed, the headrest cover 220 may be draped over the headrest of the airplane seat (adhesive strip side down) and secured to the headrest using the adhesive strips 222. It should be appreciated that the headrest cover 220 may be secured to locations other than a headrest. For example, if used for a child, the headrest cover 220 may be secured to the back of the airplane seat.

FIG. 4 illustrates an example disinfectant wipe pouch 240 and disinfectant wipe 242 of the germ shield kit of FIG. 1 according to an example embodiment. It is noted that, in one embodiment, the germ shield kit 10 may include two disinfectant wipe pouches 240 in each sealed package 200 and 202, although fewer or greater pouches may be included. The disinfectant wipe pouch 240 may be formed from a sealed, paper-backed foil package, for example, which may be torn open.

Inside each disinfectant wipe pouch 240, the disinfectant wipe 242 may be embodied as a sanitary and disinfectant wet wipe, wet towel, or moist towelette of sufficient size (e.g., 6 inch by 6 inch, 8 inch by 8 inch, etc.) for sanitizing hands and/or surfaces that an individual may come into contact with. In this context, the disinfectant wipe 242 may be used to clean and disinfect the tray table, armrest, window, or other area surrounding an airplane seat, for example. The disinfectant wipe 242 may be treated with a solution of alcohol (e.g., 70%), chlorhexidine, sporicide, or other solution (or combination of solutions) suitable as a cleaner and/or disinfectant. In some embodiments, the disinfectant wipe 242 may also be treated with softeners, lotions, or perfumes. In some embodiments, if the germ shield kit 10 includes two wipe pouches 240, the two pouches may offer different types of products. For example, a first may offer a cleaning or cleansing pad or swab and the second may offer a disinfectant wipe. In this way, an individual may use the cleansing pad to scrub away residue from the tray table, armrest, window, or other area surrounding an airplane seat, and the individual may use the disinfectant wipe to disinfect such areas.

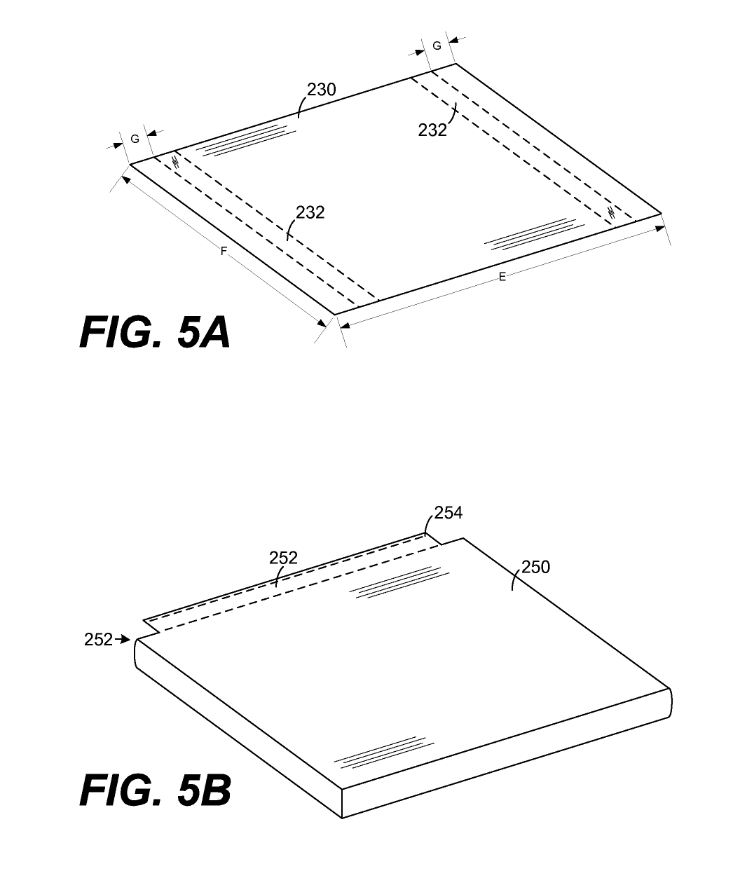

Turning to FIG. 5A, the tray cover 230 of the germ shield kit 10 of FIG. 1 is illustrated. As illustrated, the tray cover 230 may be embodied as a rectangle-shaped plastic material having length and width dimensions of E and F, respectively. In one exemplary embodiment, based on the size of an average tray on an airplane, the length E of the tray cover 230 may be selected at about 22 inches, and the width F of the tray cover 230 may be selected at about 10 inches. It should be appreciated, however, that the length and width dimensions of the tray cover 230 may vary among embodiments. The tray cover 230 may be sized to extend over the edges of an average airplane tray to provide a barrier against the edges.

The tray cover 230 may include adhesive strips 232, each positioned from the widthwise edges of the tray cover 230 by the distance G. The adhesive strips 232 may be about 1 inch wide, although other widths are within the scope of the embodiments described herein. In various embodiments, the distance G may range from ½ to 1 inch, for example. In FIG. 5A, two adhesive strips 232 along the widthwise edges of the tray cover 230 are illustrated. In other embodiments, adhesive strips may be provided along only one widthwise edge, along the lengthwise (and not widthwise) edges, along only one lengthwise (and not widthwise) edge, or along both the lengthwise and widthwise edges. The type of adhesive used for the adhesive strips 232 may be selected for suitable adherence to the plastic material of an airplane tray. The adhesive strips 232 may be relied upon to ensure a stable bond between the tray cover 230 and an airplane tray while also being easily removable, repositionable, and replaceable without leaving adhesive residue. It should be appreciated that, to the extent that the covering material of an airplane headrest is different than the plastic material of an airplane tray, the type of adhesive used for the adhesive strips 222 may be different than that used for the adhesive strips 232.

In one embodiment, the tray cover 230 may be formed from plastic sheeting having a thickness of about 2 mil (e.g., 1.5 to 2.5 mil), for example, although other thicknesses are within the scope of the embodiments described herein. Among other factors, the type, texture, and thickness of the plastic material of the tray cover 230 may be generally selected based on one or more of the following example factors: stretching ability, sheeting ability, resistance to tears and punctures, barrier properties, and folding ability. In another embodiment, the tray cover 230 may be formed from a non-woven fabric material similar to that used for the headrest cover 220. Based on its composition, the tray cover 230 may provide a fluid and particulate barrier against germs, bacteria, or other microorganisms.

In use, the tray cover 230 may be draped over an airplane tray to provide a barrier between the airplane tray and an individual's hands and arms, for example. Before being placed and secured to the airplane tray, a paper backing (e.g., waxed or slicked paper backing) is removed from the adhesive strips 232. After the paper backing is removed, the tray cover 230 may be draped over the airplane tray (adhesive strip side down) and secured to the airplane tray using the adhesive strips 232. In embodiments where the tray cover 230 is formed from a non-woven fabric material similar to that used for the headrest cover 220, the tray cover 230 may have the ability to absorb fluid, in case of spills.

While the tray cover 230 in FIG. 5A is embodied as a sheet of rectangular plastic material, the germ shield kit 10 may include other types and/or shapes of tray covers. For example, FIG. 5B illustrates a tray cover 250 of the germ shield kit 10 of FIG. 1 according to another example embodiment. As illustrated, the tray cover 250 is embodied as a sleeve of plastic material. The plastic material of the tray cover 250 may be similar to that of the tray cover 230, and the tray cover 250 may have major dimensions similar to that of the tray cover 230. However, rather than forming a drape of material, the tray cover 250 is formed as a sleeve with an opening 252 (hidden in view) at one side. Further, the tray cover 250 includes at least one flap 254 of material with an adhesive strip 252 along the flap. In use, the tray cover 250 may be slipped over an airplane tray and, using the flap 254 and adhesive strip 252, secured to a back surface or edge of the airplane tray. In other embodiments, the tray cover 250 may include two (e.g., bottom and top) flaps similar to the flap 254. In this case, a first of the two flaps may be secured to the back surface or edge of the airplane tray, and a second of the flaps may be secured over the first flap. Because the tray cover 250 is embodied as a sleeve, the tray cover 250 may provide a barrier against both top and bottom surfaces of the airplane tray.

In still other embodiments within the scope of the embodiments described herein, the tray cover may be embodied as a sheet of rectangular plastic material, with a flap and adhesive strip extending from at least a portion of each of the four edges of the tray cover. In use, each of the flaps may be secured to a respective edge (i.e., front, back, right and left) of the airplane tray.

In other aspects of the germ shield kit 10, the kit 10 may include additional barrier or comfort articles such as slippers, socks, eye covers, ear buds, or other items. Further, one or more of the articles in the germ shield kit 10 may have brand markings, logos, characters, slogans, insignias, or other markings printed or embossed upon them at various locations. Additionally, because the germ shield kit 10 may include more than one sealed package of articles (e.g., a first sealed package 200 and a second sealed package 202), certain embodiments of the germ shield kit 10 may include articles for adults in the first sealed package 100 and articles for children in the second sealed package 202. For example, the first sealed package 200 may include articles sized, branded, or otherwise prepared for adults, and the second sealed package 202 may include articles sized, branded, or otherwise prepared for children.

Although embodiments have been described herein in detail, the descriptions are by way of example. The features of the embodiments described herein are representative and, in alternative embodiments, certain features and elements may be added or omitted. Additionally, modifications to aspects of the embodiments described herein may be made by those skilled in the art without departing from the scope of the present invention defined in the following claims, the scope of which is to be accorded the broadest interpretation so as to encompass modifications and equivalent structures.

At least the following is claimed:

1. A kit of germ shield articles, comprising:
a tray cover to drape at least a top surface of a tray table of an airplane;
a headrest cover to drape a headrest on the airplane; and
at least one disinfectant wipe.

2. The kit of claim 1, further comprising an expandable bag, wherein the tray cover, the headrest cover, and the disinfectant wipe are sealed within the expandable bag.

3. The kit of claim 1, wherein the disinfectant wipe is sealed within a foil pouch.

4. The kit of claim 1, wherein the tray cover includes at least one adhesive strip.

5. The kit of claim 1, wherein the headrest cover includes at least one of an adhesive strip, a hook fastener strip, or an elastic strip.

6. A kit of germ shield articles, comprising:
a first sealed package of articles; and
a second sealed package of articles, wherein the first sealed package of articles comprises:
a tray cover;
a headrest cover of a first size; and
at least one disinfectant wipe; and
the second sealed package of articles comprises:
a tray cover;
a headrest cover of a second size; and
at least other disinfectant wipe.
